(19) United States
(12) Patent Application Publication
(10) Pub. No.: US 2010/0217004 A1
(43) Pub. Date: Aug. 26, 2010

(54) PROCESS FOR THE PRODUCTION OF TERTIARY ALCOHOLS

(76) Inventors: John McGarrity, Brig-Glis (CH); Francis Djojo, Visp (CH)

Correspondence Address: HOFFMANN & BARON, LLP, 6900 JERICHO TURNPIKE, SYOSSET, NY, 11791, US

(21) Appl. No.: 12/668,100

(22) PCT Filed: Jul. 10, 2008
(86) PCT No.: PCT/EP08/05638

§ 371 Date: Mar. 15, 2010

(30) Foreign Application Priority Data
Jul. 13, 2007 (EP) 07013809.4

(51) Int. Cl. C07D 215/18

(52) U.S. Cl. 546/180

(57) ABSTRACT

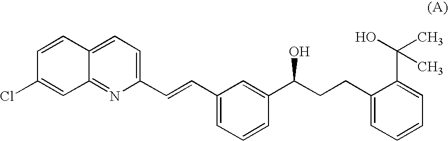

Tertiary alcohols are prepared by reacting carboxylic esters with Grignard reagents in ethereal solvents in the presence of lanthanum trichloride and lithium chloride. The method is particularly suitable for the production of (αS)-α-[3-[(1E)-2-(7-chloro-2-quino-linyl)ethenyl]phenyl]-2-(1-hydroxy-1-methylethyl)benzenepropanol of formula (A) which is an intermediate in the production of montelukast.

The invention relates to a process for the production of tertiary alcohol of formula

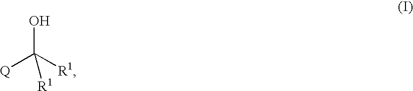

wherein R¹ is C₁₋₄ alkyl and Q is C₁₋₁₀ alkyl, C₂₋₁₀ alkenyl, C₃₋₈ cycloalkyl, aryl or heteroaryl or an organic moiety composed of any two or more of the beforementioned, each C₁₋₁₀ alkyl, C₂₋₁₀ alkenyl, C₃₋₈ cycloalkyl, aryl and heteroaryl optionally being substituted with one or more substituents independently selected from the group consisting of hydroxy, fluorine, chlorine, amino, C₁₋₄ alkylamino and di(C₁₋₄ alkyl)amino.
Tertiary alcohols having two lower alkyl groups at the carbinol carbon are valuable intermediates in the syntheses of several pharmaceutically active compounds. For example, (αS)-α-[3-[(1E)-2-(7-chloro-2-quinolinyl)ethenyl]phenyl]-2-(1-hydroxy-1-methylethyl)benzenepropanol of formula

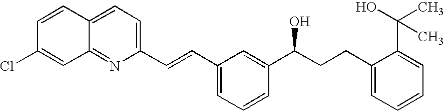

is a key intermediate in the synthesis of the pharmaceutically active compound known as montelukast (1-[[[(1R)-1-[3-[(1E)-2-(7-chloro-2-quinolinyl)ethenyl]phenyl]-3-[2-(1-hydroxy-1-methylethyl)phenyl]propyl]thio]methyl]cyclopropaneacetic acid).

A well known synthesis of tertiary alcohols is the reaction of carboxylic esters with two equivalents of a Grignard reagent. However, the yields are often not satisfactory as undesired reactions compete with the formation of the alcohol and result in the formation of byproducts, in particular when an alkylmagnesium chloride is used as Grignard reagent. It has recently been found that “nearly anhydrous” activated cerium trichloride has a beneficial effect on the above reaction, which has been postulated to be due to suppression of the enolization of the ketone intermediate (D. A. Conlon et al., Adv. Synth. Catal. 2004, 346, 1307-1315). The water content and activation method of the cerium trichloride as well as its crystal habit have been found to be critical. Moreover, the activation of the cerium chloride is somewhat tedious and the activated cerium chloride is sparingly soluble in ethereal solvents such as tetrahydrofuran which results in a heterogeneous reaction mixture. In the preparation of the above montelukast intermediate the starting material (which is available as a monohydrate) has first to be carefully dried (e.g. by azeotropic distillation), but nevertheless, about 5 equivalents of methylmagnesium chloride are required, instead of the theoretical amount of 3 equivalents (WO 95/18107 A1).

EP-A-1 759 765 discloses solutions of anhydrous lanthanide salts of formula MX₃.z LiA, such as LaCl₃.2 LiCl, and their use in Grignard-type reactions, in particular with ketones and imines. In the case of ketones, said lanthanide salts are employed in equimolar amounts and examples are given where carboxylic ester moieties are unaffected. The addition of a trace of water to the reaction mixture is said to initiate a precipitation of the lanthanide salt.

It is an object of the present invention to provide an improved method for the preparation of tertiary alcohols from carboxylic esters and Grignard reagents which gives high yields of the desired product even if the chloride form of the Grignard reagent is used and even if the starting material is used in its hydrate form. The method should not involve tedious activation steps, heterogeneous reaction mixtures and cumbersome work-up procedures.

Applicants have found that tertiary alcohols of formula

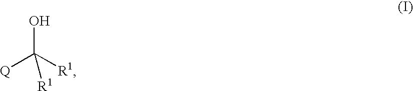

wherein R¹ is C₁₋₄ alkyl and Q is C₁₋₁₀ alkyl, C₂₋₁₀ alkenyl, C₃₋₈ cycloalkyl, aryl or heteroaryl or an organic moiety composed of any two or more of the beforementioned, each C₁₋₁₀ alkyl, C₂₋₁₀ alkenyl, C₃₋₈ cycloalkyl, aryl and heterocyclyl, optionally being substituted with one or more substituents independently selected from the group consisting of hydroxy, fluorine, chlorine, amino, C₁₋₄ alkylamino and di(C₁₋₄ alkyl)amino can be prepared by reacting a carboxylic ester of formula

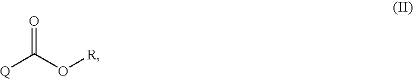

wherein R is C₁₋₁₀ alkyl, aryl or arylalkyl,
with a Grignard reagent of formula

R¹MgX (III),

wherein R¹ is as defined above and X is chlorine, bromine or iodine,
in an ethereal solvent in the presence of lanthanum trichloride and lithium chloride.

Here and hereinbelow, the term “C₁₋ₙ alkyl” is to be understood to comprise any linear or branched alkyl group having from 1 to n carbon atoms. For example, the term “C₁₋₄ alkyl” comprises methyl, ethyl, propyl, isopropyl, butyl, isobutyl, sec-butyl and tert-butyl. In addition to the beforementioned, the term “C₁₋₁₀ alkyl” comprises groups such as pentyl, isopentyl, neopentyl, hexyl, isohexyl, heptyl, octyl, nonyl, decyl and the like.

The term “C₂₋₁₀ alkenyl” comprises any linear or branched hydrocarbyl group having from 2 to 10 carbon atoms and at least one carbon-carbon double bond.
The term “C₃₋₈ cycloalkyl” is to be understood to comprise any mono- or bicyclic cycloaliphatic group having from 3 to 8 carbon atoms, such as cyclopropyl, cyclobutyl, cyclopentyl, cyclohexyl, cycloheptyl, cyclooctyl, norbornyl, norcaryl and the like.
The term “aryl” is to be understood to comprise any mono-, bi- or polycarbocyclic group comprising at least one aromatic ring, such as phenyl, naphthyl, anthracenyl, phenanthryl, biphenylyl, fluorenyl, tetrahydronaphthalenyl and the like. A preferred meaning of “aryl” is phenyl.
The term “heterocyclyl” comprises any aromatic and non-aromatic heterocyclic groups, such as tetrahydropyranyl, tetrahydrofuranyl, piperidinyl, pyrrolidinyl, morpholinyl, pyranyl, furanyl, thiophenyl, pyrrolyl, pyridyl, pyrazinyl, pyrimidinyl, oxazolyl, thiazolyl, indolyl, quinolinyl, carbazolyl and the like. Preferred meanings of “heterocyclyl” are pyridyl and quinolinyl.
The expression “organic moiety composed of any two or more of the beforementioned” is to be understood to mean any organic moiety having one free (open) valency that comprises two or more of the beforementioned groups, for example arylalkyl or alkylaryl, (arylalkyl)aryl, (arylalkenyl)aryl, [(alkenylaryl)alkyl]aryl, [[(heterocyclylalkenyl)aryl]alkyl]aryl and the like.
Each C₁₋₁₀ alkyl, C₂₋₁₀ alkenyl, C₃₋₈ cycloalkyl, aryl and heteroaryl occurring alone or as a component of an organic moiety composed of two or more of these groups, as described above, may independently be substituted with one or more substituents selected from the group consisting of hydroxy, fluorine and chlorine.

The term “ethereal solvent” is to be understood to include any solvent or solvent mixture comprising a substantial amount of an acyclic or cyclic ether that is liquid at the reaction temperature, such as diethyl ether, dibutyl ether, methyl Cert-butyl ether, dimethoxyethane, tetrahydrofuran (THF), 2-methyltetrahydrofuran, 1,4-dioxane and the like. It also includes cyclic acetals such as 1,3-dioxolane or 1,3-dioxane.

The lithium chloride solubilizes the lanthanum trichloride, resulting in a true solution of the two salts in the ethereal solvent and thus in a homogeneous reaction mixture. In a preferred embodiment, lanthanum trichloride and lithium chloride are present in a molar ratio of 1:2 or less. A THF solution of LaCl₃ and LiCl in a molar ratio of 1:2 is commercially available from Chemetall GmbH, Frankfurt (Main), Germany.

The alkyl group R¹ of the Grignard reagent III is preferably methyl.

The halogen component X of the Grignard reagent III is preferably chlorine.

In a preferred embodiment, the organic moiety Q of the tertiary alcohol I and the carboxylic ester II comprises at least one aryl group. More preferably, the carboxylate group of the carboxylic ester II is directly bound to an aryl group.

In a still more preferred embodiment, Q is the group of formula

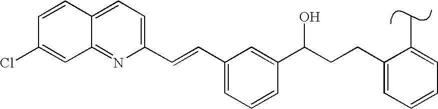

and the carboxylic ester II is

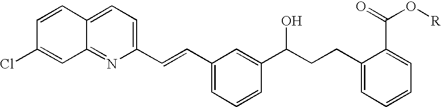

wherein R is as defined above,
to yield the tertiary alcohol I of formula

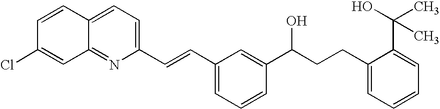

Most preferably, the secondary alcohol groups of the above structures have S-configuration to make them suitable as intermediates in the synthesis of (R)-montelukast.

In a preferred embodiment, the preferred carboxylic ester depicted above is used in the monohydrate form, thus rendering a separate drying step superfluous. The water of crystallization simply reacts with one equivalent of the Grignard reagent to yield the corresponding alkane and magnesium hydroxyhalide. This is surprising in view of EP-A-1 759 765 which stated that even traces of water initiate precipitation of the lanthanide salt.

When the monohydrate form of the ester is used as starting material, the lanthanum trichloride is advantageously used in a molar ratio of lanthanum trichloride to carboxylic ester (II) of from 1.5:1 to 1:2.

In another preferred embodiment, the preferred carboxylic ester depicted above is used in the anhydrous form which may be obtained by azeotropic dehydration of the monohydrate using a suitable entraining agent such as toluene. It has been found that it is possible to directly use the solution obtained by azeotropic removal of the water of crystallization and to add said solution to a solution comprising the Grignard reagent, the lanthanum trichloride and the lithium chloride.

When the anhydrous form of the ester is used as starting material, the amount of lanthanum trichloride can be reduced to a preferred molar ratio of lanthanum trichloride to carboxylic ester (II) of from 1:1 to 1:10, more preferably from 1:2 to 1:10 or from 1:3 to 1:10.

The starting carboxylic ester II is preferably a methyl ester.

The ethereal solvent used in the process of the invention is preferably tetrahydrofuran alone or a mixture of tetrahydrofuran and an inert solvent such as an aliphatic or aromatic hydrocarbon. Also preferred are 2-methyltetrahydrofuran and 1,3-dioxolane.

The reaction temperature can be in the range that is commonly employed in Grignard reactions, it is preferably between −20° C. and room temperature, more preferably from −10° C. to +10 ° C.

The work-up of the reaction mixture can be accomplished according to the methods commonly used in the art, e.g. by quenching with water or weak aqueous acids and extracting the product with a suitable solvent.

The following non-limiting examples will illustrate the process of the invention.

### EXAMPLE 1

### (αS)-α-[3-[(1E)-2-(7-Chloro-2-quinolinyl)ethenyl]phenyl]-2-(1-hydroxy-1-methylethyl)benzenepropanol

In a 50 mL three necked flask equipped with magnetic stirrer, a solution of lanthanum trichloride and lithium chloride (molar ratio 1:2) in THF (3.09 g of a 16 wt. % solution, 2.00 mmol) was diluted with THF (6.0 mL). Methyl 2-[(3S)-3-[3-[(1E)-2-(7-chloro-2-quinolinyl)ethenyl]phenyl]-3-hydroxypropyl]benzoate monohydrate (0.916 g, 2.00 mmol; prepared according to EP 0 480 717 A1, Example 146, Step 2) was added, and after 1 h stirring at room temperature under nitrogen the mixture was cooled to −5° C. Methylmagnesium chloride (3 M solution in THF, 3.4 mL, 10 mmol) was added dropwise while the temperature was not allowed to exceed −5° C. The mixture was then stirred at 0° C. for 12 h and after warming to room temperature saturated aqueous ammonium chloride solution (10 mL) was added at such a rate as to maintain the temperature below 25° C. Water (10 mL) and Toluene (20 mL) were added and the resulting suspension was filtered through a sintered glass filter. The phases were separated and the aqueous phase was extracted with toluene (20 mL) and discarded. The combined organic phases were washed with water (5 mL) and evaporated in vacuo (50 mbar, 40° C.) to leave a residue of 3.1 g. The residue was triturated at 50° C. with heptane (3 mL) and then cooled to 20° C. during 3 h. The precipitated product was isolated by filtration at 20° C., washed first with heptane/toluene (1:1 v/v, 4 mL), then with heptane (4 mL), and finally dried at 40° C. to yield 0.63 g of the desired product.

¹H NMR (DMSO-d₆, 500 MHz): δ=1.51 (s, 3H); 1.52 (s, 3H); 2.00 (m, 2H), 2.96 (m, 1H); 3.10 (m, 1H); 4.72 (m, 1H); 4.94 (s, 1H); 5.36 (d, J=4.4 Hz, 1H); 7.09 (t, J=7.6 Hz, 1H); 7.14 (t, J=7.8 Hz, 1H); 7.18 (d, J=6.4 Hz, 1H); 7.41 (m, 2H); 7.44 (d, J=7.9 Hz, 1H); 7.49 (d, J=16.6 Hz, 1H); 7.56 (dd, J=8.3, 2.2 Hz, 1H); 7.62 (d, J=6.8 Hz, 1H); 7.77 (bs, 1H); 7.91 (d, J=16.6 Hz, 1H); 7.92 (d, J=8.7 Hz, 1H); 7.99 (d, J=8.8 Hz, 1H); 8.03 (d, J=2.0 Hz, 1H); 8.38 (d, J=8.4 Hz, 1H).
¹³C NMR (DMSO-d₆, 126 MHz): δ=29.82, 31.55, 31.57, 42.34, 71.60, 72.31, 120.24, 124.73, 124.88, 125.24, 125.51, 125.71, 126.22, 126.54, 127.16, 128.04, 128.49, 129.65, 130.82, 134.23, 135.20, 135.67, 136.43, 140.25, 146.66, 146.93, 147.99, 156.78.

### EXAMPLES 2-4

The procedure of Example 1 was repeated using different amounts of lanthanum trichloride (0.5, 1.0 and 1.5 molar equivalents) and 10 molar equivalents of methylmagnesium chloride (instead of 5 molar equivalents). The yield of the desired product was determined by HPLC.

The observed yields were as follows:
0.5 equivalents LaCl₃: 88.3%
1.0 equivalents LaCl₃: 94.9%
1.5 equivalents LaCl₃: 98.5%

### EXAMPLE 5

### (αS)-α-[3-[(1E)-2-(7-Chloro-2-quinolinyl)ethenyl]phenyl]-2-(1-hydroxy-1-methylethyl)benzenepropanol

In a first 500 mL reaction vessel a 14.7 wt. % solution of LaCl₃/LiCl in THF (14.03 g, 8.4 mmol, 0.2 equiv) was diluted with THF (30 mL). A 3.0 M solution of MeMgCl (71.43 g, 210 mmol, 5 equiv) was added to the solution at room temperature to obtain a first reaction mixture. The solution was cooled to −9° C. In a second 500 mL reaction vessel a mixture of methyl 2-[(3S)-3-[3-[(1E)-2-(7-chloro-2-quinolinyl)ethenyl]phenyl]-3-hydroxypropyl]benzoate monohydrate (20.0 g, 42.02 mmol, 1 equiv) was added to toluene (200 mL). Water in the reaction mixture was removed by azeotropic distillation (50° C., 100 mbar) until the volume was reduced to 60 mL. THF (40 mL) was added to the distillation residue in order to obtain a clear solution. Subsequently, the solution was cooled to 20° C. and transferred into the first reaction mixture while keeping the internal temperature of the first reaction vessel in the range of −9° C. to −5° C. The reaction mixture was allowed to stand for an additional 1.5 h while the reaction progression was monitored by HPLC. After completion of the reaction the solution was cooled to −15° C. and was quenched by addition of 4 M aqueous acetic acid (128 mL) while keeping the internal temperature below 10° C. The resulting biphasic system was kept to 10° C. Toluene (50 mL) was added and the system was stirred for 15 min at 10° C., settled for 5 min at 10° C. to give a clear two phase separation. The organic layer was then separated and washed with a 10 wt. % solution of Na₂CO₃ (104 mL) at 10° C. and with a 10 wt. % solution of NaCl (104 mL) at 10° C. The organic phase was concentrated (to 60 mL) in vacuo (40° C., 150 mbar). The distillation residue was heated to 60° C. and heptane (15 g) was added over 10 min followed by seeding with crystals of the desired product in order to initiate crystallisation at 60° C. The suspension was stirred for an additional 2 h at 60° C. Heptane (60 g) was added over 10 h at 60° C. The suspension was cooled over 1 h to 0° C. and the precipitated product was isolated on a glass filter funnel, washed with heptane (50 mL) at 20° C. and vacuum dried at 45° C. The drying was monitored by Karl Fischer titration.

Yield: 18.3 g (94.1%) of dry product (assay: 98.7%).

### EXAMPLE 6

### 2-Phenyl-2-propanol

A 13.9 wt. % solution of LaCl₃/LiCl in THF (14.84 g, 8.4 mmol, 0.2 equiv) was diluted with THF (50 mL). A 3.0 M solution of methylmagnesium chloride (42.86 g, 126 mmol, 3 equiv) was added to the solution at room temperature. The solution was then cooled to −9° C. and a mixture of ethyl benzoate (6.30 g, 42.9 mmol, 1 equiv) in toluene (7 mL) was added to the first solution over 60 min within a temperature range of −9° C. to −5° C. After an additional 30 min an in-process control with GLC showed no starting material present. The reaction mixture was cooled to −20° C. and was quenched by addition of 4 M aqueous acetic acid (128 mL) while the temperature was kept below 10° C. The resulting biphasic system was allowed to warm to 20° C. Toluene (50 mL) was added and the system was agitated for 15 min at 20° C. and settled for 5 min at 20° C. to give a clear phase separation. The organic layer was washed at 20° C. with a 10 wt. % aqueous Na₂CO₃ solution (104 mL), followed by a 10 wt. % aqueous NaCl solution. Then the organic phase was dried over Na₂SO₄ and concentrated in vacuo to give the product as a yellow oil. The structure of the product was confirmed by ¹H NMR.

Yield: 89.7%, purity (GLC): 98%.

### EXAMPLE 7

### 2-Phenyl-2-propanol

The procedure of Example 6 was repeated using 5 equivalents of MeMgCl.

The isolated yield was 93.9% with 99.6% purity.

### EXAMPLE 8

### 2-Methylundecan-2-ol

The procedure of Example 6 was repeated using methyl decanoate instead of ethyl benzoate.

The isolated yield was 76.6% with 97.6% purity.

### EXAMPLE 9

### (αS)-α-[3-[(1E)-2-(7-Chloro-2-quinolinyl)ethenyl]phenyl]-2-(1-hydroxy-1-methylethyl)benzenepropanol

Methyl 2-[(3S)-3-[3-[(1E)-2-(7-chloro-2-quinolinyl)ethenyl]phenyl]-3-hydroxypropyl]benzoate monohydrate (20.00 g, 42.01 mmol) and 2-methyltetrahydrofuran (100 mL) were charged to a 250 mL reactor. To remove water, 60 mL of solvent was distilled from the solution at 79° C. and normal pressure. Karl Fischer analysis showed that the water content was 0.04% (1 mL of solution was drawn). The solution was stored at 20-30° C. Methylmagnesium chloride (59.04 g, 175.24 mmol) was charged under nitrogen to a second 250 mL reactor and cooled to −10° C. Then, 14.10 g of a 16.06 wt. % solution of LaCl₃.2 LiCl in tetrahydrofuran was added within 0.5 h. The resulting suspension was cooled to −15° C. The solution in the first reactor was syringed into the second reactor at such a rate as to maintain the temperature below −10° C. The reaction was monitored by HPLC. After the reaction was completed, 4 M acetic acid (90 mL) was added slowly, while maintaining the temperature below 0° C. (pH of water phase: 5-6). The mixture was heated to 20° C. The organic phase was separated and washed twice with 10 wt. % aqueous Na₂CO₃ (90 mL) and twice with 10 wt. % aqueous NaCl (60 mL each). To remove water and tetrahydrofuran, 15.0 g of solvent was distilled from above solution. Then 2-methyltetrahydrofuran (32.0 g) was added, followed by distillation of another 24.0 g of solvent. The residue was cooled to 30° C. and then n-heptane (22.4 g) was added to form a saturated solution. To the solution, 0.4 g of the diol product was added as seed crystals and the resulting suspension was stirred overnight. n-Heptane (75.0 g) was added within 1.5 h, and the suspension was cooled to −2° C. within 1 h and kept at this temperature for 3 h. The product was isolated by filtration. The filter cake was washed with n-heptane (30 mL) and dried at 40° C./<100 mbar.

Yield: 17.5 g (87%), purity 98.4% (ketone content 0.6%).

### EXAMPLE 10

### (αS)-α-[3-[(1E)-2-(7-Chloro-2-quinolinyl)ethenyl]phenyl]-2-(1-hydroxy-1-methylethyl)benzenepropanol

Methyl 2-[(3S)-3-[3-[(1E)-2-(7-chloro-2-quinolinyl)ethenyl]phenyl]-3-hydroxypropyl]benzoate monohydrate (10.0 g, 21.62 mmol) followed by 1,3-dioxolane (50 mL) were charged to a 100 mL reactor. To remove water, 30 mL of solvent was distilled from the solution at 79° C. and normal pressure. Karl Fischer analysis showed that water content was 0.07% (1 mL of solution was drawn). The solution was stored at 20-30° C. Methylmagnesium chloride (30.10 g, 89.34 mmol) was charged under nitrogen to a 250 mL reactor and cooled to −10° C. Then LaCl₃/LiCl solution in THF (16.06 wt. %, 7.75 g) was added within 0.5 h. The suspension was cooled to −15° C. The solution in the first reactor was syringed into the second reactor at such a rate as to maintain the temperature below −10° C. The reaction was monitored by HPLC. After the reaction was completed, 4 M acetic acid (40 mL) was added slowly, while maintaining the reaction mixture below 0° C. (pH value of water phase: 5-6). The organic phase was separated and washed with aqueous Na₂CO₃ (5 wt. %, 30 mL) and NaCl (10 wt. %, 30 mL). The solvent was removed in vacuo and then the residue was dissolved in toluene (10 mL). The solution was heated to 45° C. and n-heptane (3 mL) was added. Seed crystals of the desired product (0.4 g) were added to induce crystallization. The suspension formed was heated to 50° C. and stirred for 3 h. n-Heptane (30 mL) was added within 3 h, followed by cooling to 0° C. The product was isolated by filtration. The filter cake was washed with n-heptane (15 mL) and dried at 40° C./<100 mbar.

Yield: 8.9 g (83.3%), purity 98.4% (ketone content 0.6%).

1. A process for the production of tertiary alcohols of formula 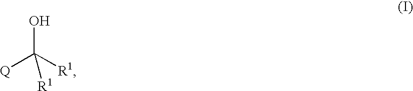
wherein R¹ is C₁₋₄ alkyl and Q is C₁₋₁₀ alkyl, C₂₋₁₀ alkenyl, C₃₋₈ cycloalkyl, aryl or heteroaryl or an organic moiety composed of any two or more of the beforementioned, each C₁₋₁₀ alkyl, C₂₋₁₀ alkenyl, C₃₋₈ cycloalkyl, aryl and heteroaryl, optionally being substituted with one or more substituents independently selected from the group consisting of hydroxy, fluorine, chlorine, amino, C₁₋₄ alkylamino and di(C₁₋₄ alkyl)amino, by reacting a carboxylic ester of formula 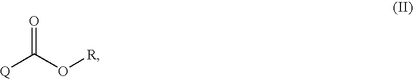
wherein R is C₁₋₁₀ alkyl, aryl or arylalkyl,
with a Grignard reagent of formula
R¹MgX (III),
wherein R¹ is as defined above and X is chlorine, bromine or iodine,
in an ethereal solvent in the presence of lanthanum trichloride and lithium chloride.

2. The process of claim 1, wherein lanthanum trichloride and lithium chloride are present in a molar ratio of 1:2.

3. The process of claim 1 or 2, wherein R¹ is methyl.

4. The process of any of claims 1 to 3, wherein X is chlorine.

5. The process of any of claims 1 to 4, wherein Q comprises an aryl group.

6. The process of claims 4 and 5, wherein Q is the group of formula 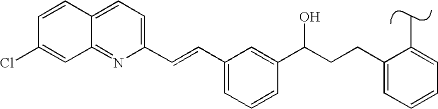
and the carboxylic ester II is 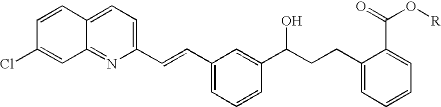
wherein R is as defined in claim 1,
to yield the tertiary alcohol I of formula

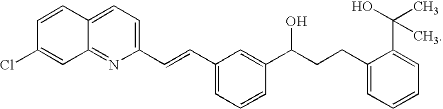

7. The process of claim 6, wherein the secondary alcohol group in Q has S-configuration.

8. The process of claim 6 or 7, wherein the carboxylic ester is used in the monohydrate form.

9. The process of claim 8, wherein the molar ratio of lanthanum trichloride to carboxylic ester (H) is from 1.5:1 to 1:2.

10. The process of claim 6 or 7, wherein the carboxylic ester is used in the anhydrous form.

11. The process of claim 10, wherein the molar ratio of lanthanum trichloride to carboxylic ester (II) is from 1:1 to 1:10.

12. The process of claim 11, wherein the molar ratio of lanthanum trichloride to carboxylic ester (II) is from 1:2 to 1:10.

13. The process of any of claims 1 to 11, wherein R is methyl.

14. The process of any of claims 1 to 12, wherein the ethereal solvent is selected from tetrahydrofuran, 2-methyltetrahydrofuran, 1,3-dioxolane, and mixtures of the beforementioned with an inert solvent.
